(19) United States
(12) Patent Application Publication
(10) Pub. No.: US 2004/0122392 A1
(43) Pub. Date: Jun. 24, 2004

(54) Contact-dry article

(76) Inventor: Charishma Mohini W. Seneviratne, Como (AU)

Correspondence Address: Michealson & Associates, Michaelson & Wallace, Parkway 109 Office Center P O Box 8489, 328 Newman Springs Road, Red Bank, NJ, 07701, US

(21) Appl. No.: 10/474,478

(22) PCT Filed: Apr. 8, 2002
(86) PCT No.: PCT/AU02/00441

(30) Foreign Application Priority Data
Apr. 9, 2001 (AU) PR 4315

(51) Int. Cl. A61F 13/15; A61F 13/20

(52) U.S. Cl. 604/367; 604/378

(57) ABSTRACT

A contact-dry article that acts to absorb body liquid. In the form of nappy pads, panty liners, trainer pants, bed liners and surgeon's underwear. The contact-dry article (10) has a first liquid pervious layer (1) to be placed in contact with a user and recive liquid deposited thereon, a second liquid pervious layer (2), a bulk absorbent layer (4) to receive and dissipate liquid therewithin and a liquid impervious layer (7). Optionally, the contact-dry article (10) further comprises a first absorbent/drying layer (3) to receive liquid from the second liquid pervious layer (2), a second absorbent/drying layer (5) to receive excess liquid from the bulk absorbent layer (4), a bulk drainer layer (6) between the second absorbent/drying layer (5) and the liquid impervious layer (7) and a third liquid pervious layer (8) to cover the liquid impervious layer (7).

### FIELD OF THE INVENTION

The present invention relates to a contact-dry article.

The contact-dry article of the present invention is intended to be placed in contact with the skin of a user, i.e. a person, and acts to absorb body liquids that may be deposited on the contact-dry article.

### DISCLOSURE OF THE INVENTION

In accordance with one aspect of the present invention there is provided a contact-dry article comprising;

a first liquid pervious layer, having a surface to be placed in contact with a user, to receive liquid deposited thereon,

a second liquid pervious layer to receive liquid from said first liquid pervious layer,

a bulk absorbent layer to receive and dissipate liquid therewithin, the liquid being received from said second liquid pervious layer, and

a liquid impervious layer to resist passage of liquid therethrough.

Preferably, said contact-dry article further comprises a first absorbent drying layer to receive liquid from said second liquid pervious layer and said first absorbent/drying layer is provided between said second liquid pervious layer and said bulk absorbent layer.

In accordance with another aspect of the present invention there is provided a contact-dry article comprising:

a first liquid pervious layer, having a surface to be placed in contact with a user, to receive liquid deposited thereon,

a second liquid pervious layer to receive liquid from said first liquid pervious layer,

a first absorbent/drying layer to receive liquid from said second liquid pervious layer,

a bulk absorbent layer to receive and dissipate liquid therewithin, the liquid being received from said first absorbent/drying layer,

a second absorbent drying layer to receive excess liquid from said bulk absorbent layer,

a liquid impervious layer to resist passage of liquid therethrough.

Preferably, a bulk drainer layer is provided between said second absorbent/drying layer and said liquid impervious layer.

Preferably, a third liquid pervious layer is provided to cover said liquid impervious layer.

Preferably, said first liquid pervious layer and said third liquid pervious layer are made of similar material.

Preferably, said second liquid pervious layer is resistant to passage of liquid therefrom back to said first liquid pervious layer.

Preferably, said first liquid pervious layer, said second liquid pervious layer, said third liquid pervious layer and said bulk drainer layer comprise cellular fibres and/or hydrophobic fibres.

It is to be understood that references herein to “hydrophobic” fibres, yarn, layers, fabrics, and the like, are to be understood as including such materials that can retain minimal amount of liquid and still be considered to be hydrophobic. Such hydrophobic materials can have varying capacities of liquid absorption or retention, though there would be at a minimum.

Preferably, said first absorbent/drying layer and said second absorbent/drying layer comprise cellular fibres and/or hydrophobic fibres.

Preferably, said first liquid pervious layer, said second liquid pervious layer, said third liquid pervious layer and said bulk drainer layer each comprise substantially from 50% to 100% cellular fibres.

Preferably, said cellular fibres comprise substantially from 35% to 85%.

Preferably, said cellular fibres comprise cotton and/or hemp.

Preferably, said first liquid pervious layer, said second liquid pervious layer, said third liquid pervious layer and said bulk drainer layer each comprise substantially from 10% to 100% hydrophobic fibres.

Preferably, said hydrophobic fibres comprise substantially from 65% to 100%.

Preferably, said hydrophobic fibres comprise polyester and/or acrylic.

Preferably, said first liquid pervious layer and said third pervious layer are provided as a knitted fabric.

Preferably, said second liquid pervious layer is provided as a perforated fabric.

Preferably, said first absorbent/drying layer and said second absorbent/drying layer comprise coton.

Preferably, said bulk absorbent layer comprises compressible web material.

Preferably, said bulk absorbent layer comprises terry towelling.

Preferably, said liquid impervious layer comprises water-proof coated fabric.

The contact-dry article of the present invention may be provided as an article from which another article is made, or may itself be provided as such another article. By way of example, such other articles include, but without limitation, nappy pads, panty liners, trainer pants, incontinent pads, bed liners, bed protectors and surgeon's underwear.

### BRIEF DESCRIPTION OF THE DRAWINGS

The present invention will now be described, by way of example, with reference to the accompanying drawings, in which:

FIG. 1 is an exploded view of the layers of an embodiment of a contact-dry article in accordance with the present invention, in which the passage of moisture through and between the layers is shown by arrows;

FIG. 2 is a plan view of an embodiment of a contact-dry article of the present invention provided as an infant's bed protector;

FIG. 3 is a top view of a first face of an embodiment of a contact-dry article of the present invention provided as a pad that can be incorporated into a nappy;

FIG. 4 is a view of the other face of the nappy pad shown in FIG. 3;

FIG. 5 shows the arrangement of the nappy pad, shown in FIGS. 3 and 4, when incorporated in a nappy that can be positioned on the body of an infant;

FIG. 6 is a view of the reverse face of an embodiment of a contact-dry article of the present invention provided as a pad;

FIG. 7 shows the pad of FIG. 6 releasably attached to an infant's panty;

FIG. 8 shows a front view of the infant's panty incorporating the pad shown in FIG. 6:

FIG. 9 shows the pad of FIG. 6, in a folded condition for packaging.

### BEST MODE(S) FOR CARRYING OUT THE INVENTION

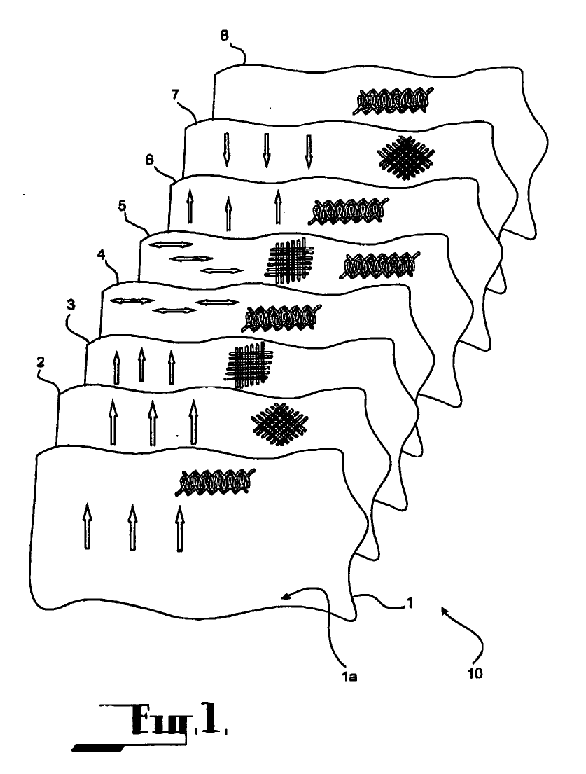

FIG. 1 shows th layers of a contact-dry article 10 in accordance with the present invention.

The contact-dry article 10 of the present invention absorbs liquids that are deposited on a surface thereof and draws them away from the surface. The surface is arranged to be placed in contact with a user. Accordingly, drawing the liquid away from the surface results in a comparatively dry, i.e. contact-dry, surface of the article 10 remaining in contact with the user. The liquid being drawn away from the surface means that the surface is relatively dry, i.e. free of moisture.

The description that follows is of an embodiment of a contact-dry article 10 in the form of a composite layer article.

The contact-dry article 10 shown in FIG. 1 comprises a first liquid pervious layer 1 having a surface 1a to be placed in contact with a user, a second liquid pervious layer 2 to receive liquid from the first liquid pervious layer 1, a first absorbent/drying layer 3 to receive liquid from the second liquid pervious layer 2, a bulk absorbent layer 4 to receive and dissipate liquid therewithin, a second absorbent/drying layer 5 to receive excess liquid from the bulk absorbent layer 4, and a liquid impervious layer 7 to resist passage of liquid therethrough.

The contact-dry article 10 shown in FIG. 1 also comprises a bulk drainer layer 6 provided between the second absorbent/drying layer 5 and the liquid impervious layer 7, and a third liquid pervious layer 8 provided to cover the liquid impervious layer 7.

In use of the contact-dry article 10, the third liquid pervious layer 8 is fartherest, from the skin of the user of the contact-dry article 10, and forms the outermost layer of the contact-dry article 10.

The surface 1a of the first liquid pervious layer 1 is shown uppermost in FIG. 1.

The layers 1-8 previously hereinbefore described are provided sequentially adjacent one another. However, the bulk drainer layer 6 and the third liquid pervious layer 8 are optional and therefore either one, or both, may be omitted. In the case of the bulk drainer layer 6 being omitted, the second absorbent/drying layer 5 is adjacent the liquid impervious layer 7. In the case of the third liquid pervious layer 8 being omitted, the liquid impervious layer 7 forms the outermost layer of the contact-dry article 10.

In an alternative form of a contact-dry article in accordance with the present invention, the first absorbent/drying layer 3 and the second absorbent/drying layer 5 are omitted. In other respects, this alternative form of the contact-dry article is the same as the one that does incorporate the first and second absorbent/drying layers 3 and 5. This alternative form of the contact-dry article is particularly suited when provided as an article for premature infants from birth up to about 6 months following birth. Omission of the first and second absorbent/drying layers 3 and 5 reduces the bulk of the contact-dry article making it more suitable for small infants born prematurely, in view of their small size.

However, in some applications of the alternative form of the contact-dry article it may be desirable to incorporate one of the first or second absorbent/drying layers 3 and 5. Generally, it is preferable to incorporate the first absorbent/drying layer 3, which is located between the second liquid pervious layer 2 and the bulk absorbent layer 4.

The first liquid pervious layer 1 and the third liquid pervious layer 8 may be made of similar material.

The second liquid pervious layer 2 is resistant to transfer of liquid therefrom back to the first liquid pervious layer 1.

The first, second and third liquid pervious layers 1, 2 and 8 and the bulk drainer layer 6 comprise cellular fibres and/or hydrophobic fibres. The cellular fibres may comprise 50% to 100% of these layers whilst the hydrophobic fibres comprise 10% to 100%, with the relative percentages totalling 100%, or less. The total may be less than 100% in cases where small quantities of some other substance, or substances, are desirably included. The most preferred content is 35% to 85% cellular fibres and 65% to 100% hydrophobic fibres.

The use of cellular fibres and hydrophobic fibres provides a material with good liquid absorbency properties (due to the cellular fibres) and strength (due to the hydrophobic fibres).

The first liquid pervious layer 1 and th third liquid pervious layer 8 may each comprise a double knitted fabric.

A knitted fabric allows flow of air therethrough. This enhances the comfort of the user.

The second liquid pervious layer 2 may comprise a perforated fabric, such as a woven fabric.

The bulk drainer layer 6 may comprise a padding, and may be a knitted fabric.

The cellular fibres comprise cotton and/or hemp and the hydrophobic fibres comprise polyester and/or acrylic, though small quantities of some other substance, or substances, may be included if that is considered desirable.

Whilst the first liquid pervious layer 1 allows the liquid that is deposited thereon to pass therethrough to the second liquid pervious layer 2, the second liquid pervious layer 2 rests passage of liquid back to the first liquid pervious layer 1. The second liquid pervious layer 2 allows liquid to pass to the first absorbent/drying layer 3.

The first absorbent/drying layer 3 is able to absorb liquid transferred thereto from the second liquid pervious layer 2 and has a relatively fast drying time.

The second liquid pervious layer 2 does not retain liquid that passes thereto from the first liquid pervious layer 1. Instead, the second liquid pervious layer 2 acts to transfer liquid, that it has received from the first liquid pervious layer 1, to the first absorbent/drying layer 3. This, in combination with the ability of the second liquid pervious layer 2 to resist passage of liquid back to the first liquid pervious layer 1, results in liquid being drawn away from the surface 1a, on which the liquid was deposited, of the first liquid pervious layer 1. This keeps the first liquid pervious layer 1, which is, in use, next to the body of the user, dry.

The first absorbent/drying layer 3 is made of cellular and/or polymer fibres, e.g. cotton and/or polymers.

The first absorbent/drying layer 3 may comprise a woven fabric.

The bulk absorbent layer 4 receives liquid from the first absorbent/drying layer 3. The bulk absorbent layer 4 is able to dissipate this liquid within itself.

The material of the bulk absorbent layer 4 allows ventilation and airflow therethrough and evaporation of the liquid absorbed therein due to body heat of the user and ventilation.

The bulk absorbent layer 4 varies in size depending upon the nature of the article 10 and the extent of the ability of the bulk absorbent layer 4 to absorb and dissipate liquid received from the first absorbent/drying layer 3.

The bulk absorbent layer 4 is a compressible web material.

The bulk absorbent layer 4 may comprise a terry towelling material and/or a knitted fabric.

The second absorbent/drying layer 5 is able to receive liquid from the bulk absorbent layer 4. Thus, any excess liquid from the bulk absorbent layer 4 can be absorbed by the second absorbent/drying layer 5. This prevents the bulk absorbent layer 4 becoming filled with liquid. The second absorbent/drying layer 5 is made of material similar to the material of the first absorbent/drying layer 3 and has similar properties.

In the alternative form of the contact-dry article 10, previously hereinbefore described, when the first and second absorbent/drying layers 3 and 5 are omitted, liquid passes from the second liquid pervious layer 2 directly to the bulk absorbent layer 4, and from the bulk absorbent layer 4 directly to the bulk drainer layer 6.

Similarly, in the event that the first absorbent/drying layer 3 is provided, but the second absorbent/drying layer 5 is omitted, the liquid passes from the second liquid pervious layer 2 to the first absorbent/drying layer 3 and then to the bulk absorbent layer 4, as previously hereinbefore described.

The liquid impervious layer 7 is provided to contain liquid and moisture within the article 10 and resist leakage from the other layers of the article 10.

The liquid impervious layer 7 may be made of waterproof coated fabric.

The bulk drainer layer 6 is optionally provided to ensure that in the event there is any excess liquid that passes beyond the second absorbent/drying layer 5, it is absorbed by the bulk drainer layer 6.

The third liquid pervious layer 8 is provided to cover the liquid impervious layer 7. The third liquid pervious layer 8 is made of material similar to the material of the first liquid pervious layer 1. The third liquid pervious layer 8 provides a “soft touch” fabric outer layer for the contact-dry article 10.

The drawings illustrate the nature of the materials, i.e. woven or knitted, from which the various layers of the contact-dry article 10 are made.

The first liquid pervious layer 1 and third liquid pervious layer 8 may each have a density of 100 gm⁻²; the second pervious layer 2 may have a density of 50 gm²; the first absorbent/drying layer 3 and the second absorbent drying layer 5 may each have a density of 85 gm⁻²; the bulk absorbent layer 4 may have a density of 125 gm⁻²; the bulk drainer layer 6 may have a density of 50 gm⁻²; and the liquid pervious layer 7 may have a density of 50 gm⁻².

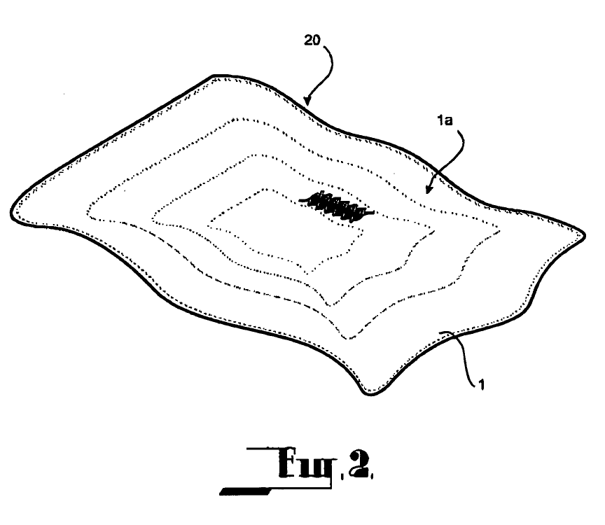

FIG. 2 shows an infants bed protector, or bed liner 20, in accordance with the present invention. The bed protector 20 is a sheet material of composite layer structure as previously hereinbefore described with reference to FIG. 1. In use, the bed protector 20 is positioned to underlie normal bed sheeting, with the surface 1A of the first liquid pervious layer 1 of the bed protector 20 located uppermost.

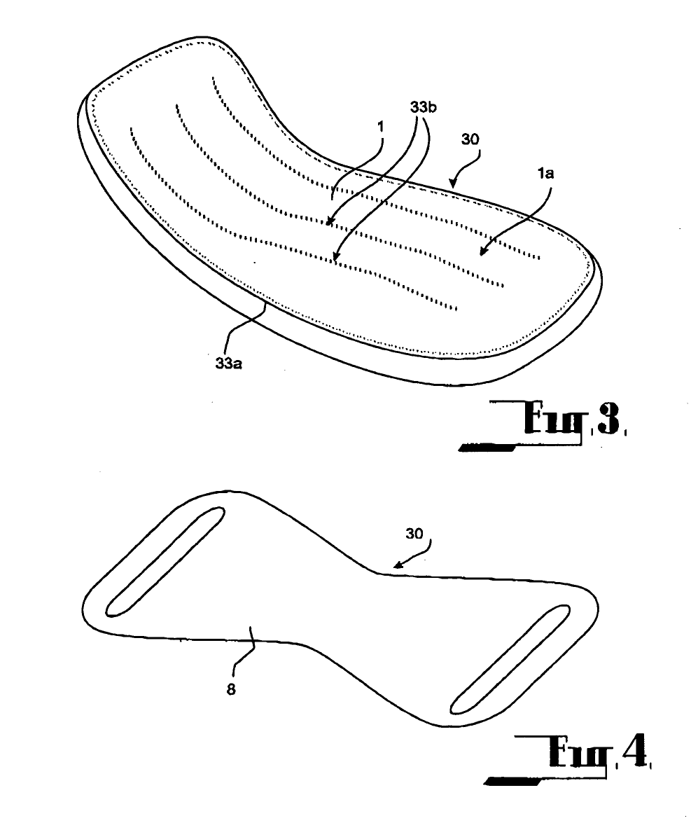

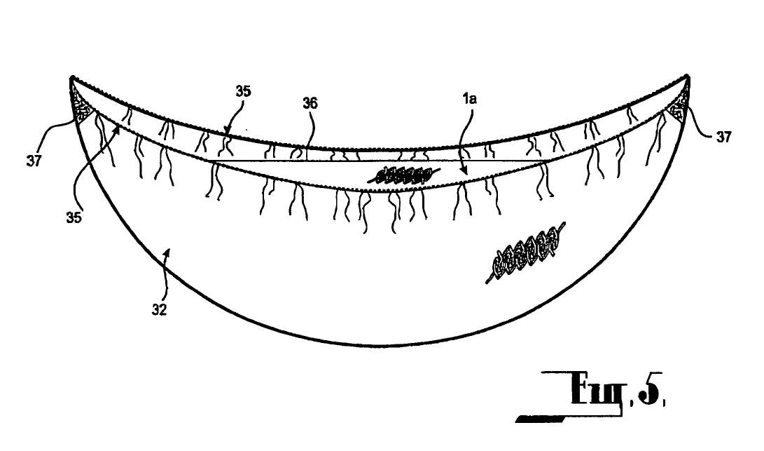

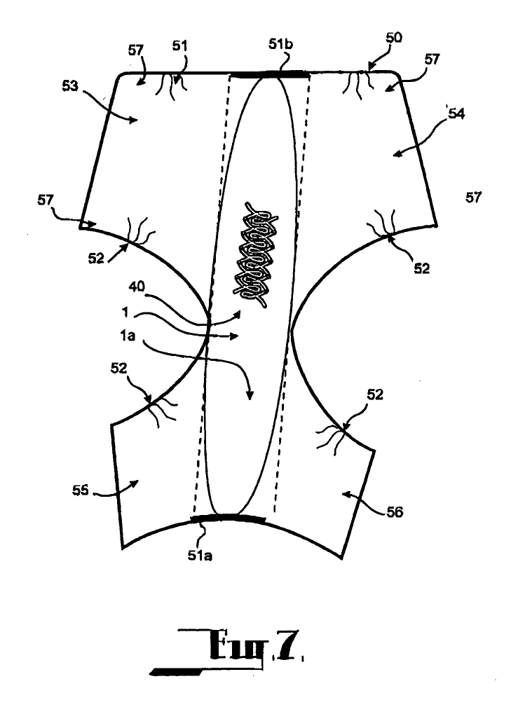

FIGS. 3 and 4 show an embodiment of the article 10 of the present invention in the form of a pad 30 that can be incorporated into an infant's nappy 32 (shown in FIG. 6). The pad 30 is provided celly in the nappy 32.

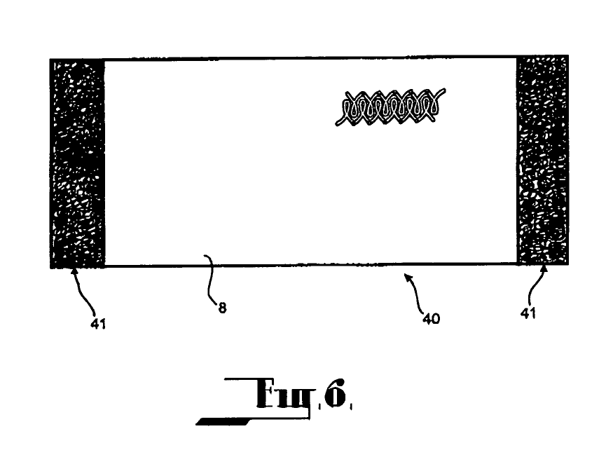

FIG. 5 shows the shape adopted by the nappy 32, in use, positioned on the body of an infant. This is a cradle-shape and takes the weight of fluid in the pad 30.

Attachment means, such as hook and loop fasteners 37, may be provided so that the ends of the nappy 32 may be attached together to retain the nappy 32 on the body of the infant.

The surface 1a of the first liquid pervious layer 1 is positioned uppermost on the nappy 32 so that it is in contact with the skin of the infant wearing the nappy 32.

FIG. 6 shows an embodiment of a pad 40 having the composite layer structure as hereinbefore described with reference to FIGS. 1 and 2 for releasable attachment to a nappy. The attachment may be by fasteners which are provided on the (outermost) third liquid pervious layer 8 of the pad 40.

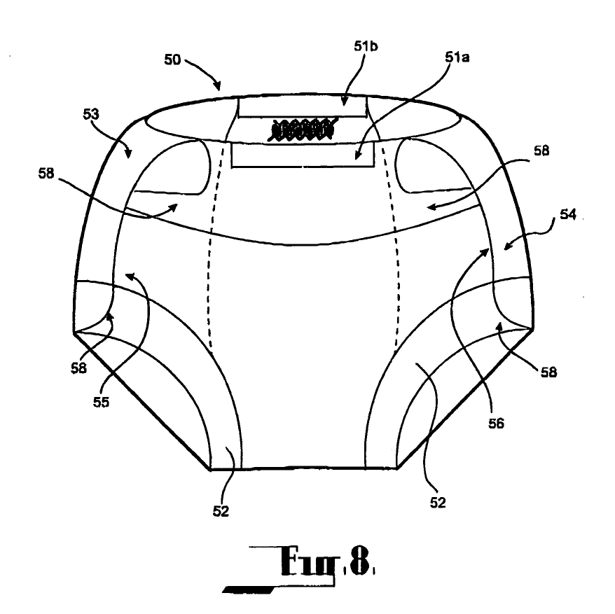

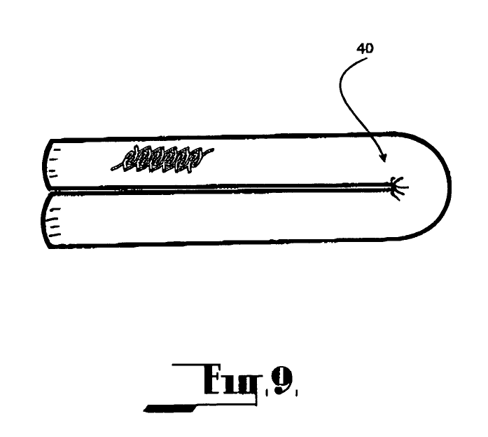

The pad 40 may then be attached to the crotch area of a conventional nappy or an infant's panty 50, i.e. an infant's underpants, as shown in FIGS. 7 and 8.

The pad 40 is attached to the panty 50 by the hook fasteners 41. The panty 50 may be provided with corresponding loop fasteners (not shown) which engage with the hook fasteners 41.

The panty 50 has an elasticised waist back 51 and elasticised edges 52, which form. In use, leg openings for the infant. The front and rear edges of the panty 50 may be provided with a soft material edge, e.g. such as satin, as shown at reference numerals 51a and 51b. This enhances comfort of the infant.

FIG. 8 shows the panty 50 in its arrangement when worn by an infant. First and second rear portions 53 and 54 of the panty 50 fold around to the front of the panty 50 and overlie front portions 55 and 56 of the panty 50. The rear portions 53 and 64 are attached to the front portions 55 and 56 by way of hook and loop fasteners 57 and 58 carried by the rear portions 53 and 54 and the front portions 55 and 56, respectively. The shape of the panty 50 may differ, for convenience, as required.

The panty 50 may be made of bleached or unbleached polyester/cotton material.

FIG. 9 shows the pad 40 in a folded condition which enables ready packaging thereof.

Whilst specific embodiments of the contact-dry article of the present invention have been described with reference to FIGS. 2 to 9, it is to be understood that the present invention can be embodied as other articles.

The contact-dry article of the present invention may also be embodied in nappies which are suitable for infants, ranging from premature infants to full grown babies up to 24 months. The weights of such premature babies would generally be in the range from under 1 kg to 1.4 kg. Older infants would weigh in a range from about 8 kg to 10 kg.

Preferably, all edges and centre sealing of the contact-dry article according to the present invention are pressure pressed by heat. This ensures nonleakage of the liquid from any joins. This is exemplified in FIG. 3 in which pressure sealing of the edges 33a and contre sealing 33b is shown.

Articles made that incorporate the contact-dry article according to the present invention may be reusable. Due to the materials from which the articles are constructed, they may be resistant to causing infection, rash and eczema in the user due to the fabric ventilation incorporated into the construction. This is particularly advantageous when the article is a nappy or nappy pad to be worn by an infant as an infant's skin is much more sensitive than that of an adult.

The articles made that incorporate the contact-dry article according to the present invention may also be washable, fast drying and hygienic.

Modification and variations such as would be apparent to a skilled addressee are deemed to be within the scope of the present invention.

Throughout the specification, unless the context requires otherwise the word “comprise” or variations such as “comprises” or “comprising”, will be understood to imply the inclusion of a stated integer or group of integers but not the exclusion of any other integer or group of integers.

1. A contact-dry article characterized in that it comprises:
a first liquid pervious layer, having a surface to be placed in contact with a user, to receive liquid deposited thereon,
a second liquid pervious layer to recive liquid from said first liquid pervious layer,
a bulk absorbent layer to receive and dissipate liquid therewithin, the liquid being received from said second liquid pervious layer, and
a liquid impervious layer to resist passage of liquid therethrough.

2. A contact-dry article according to claim 1, characterized in that it further comprises a first absorbent/drying layer to receive liquid from said second liquid pervious layer and said first absorbent drying layer is provided between said second liquid pervious layer and said bulk absorbent layer.

3. A contact-dry article according to claim 1 or 2, characterized in that it further comprises a second absorbent/drying layer to receive excess liquid from said bulk absorbent layer.

4. A contact-dry article according to claim 3, characterized in that it further comprises a bulk drainer layer provided between said second absorbent/drying layer and said liquid impervious layer.

5. A contact-dry article, according to any one of claims 1 to 4, characterized in that it further comprises a third liquid pervious layer provided to cover said liquid impervious layer.

6. A contact-dry article according to claim 5, characterized in that said first liquid pervious layer and said third liquid pervious layer are made of similar material.

7. A contact-dry article according to any one of claims 1 to 6, characterized in that said second liquid pervious layer is resistant to passage of liquid therefrom back to said first liquid pervious layer.

8. A contact-dry article according to any one of claims 1 to 7, characterized in that said first liquid pervious layer and said second liquid pervious layer comprise cellular fibres and/or hydrophobic fibres.

9. A contact-dry article according to any one of claim 4, or claims 6 to 8 when appended directly or indirectly to claim 4, characterized in that said bulk drainer layer comprises cellular fibres and/or hydrophobic fibres.

10. A contact-dry article according to any one of claims 5 or 6, or claims 7 to 9 when appended directly or indirectly to claim 5 or 6, characterized in that said third liquid pervious layer comprises cellular fibres and/or hydrophobic fibres.

11. A contact-dry article according to any one of claims 1 to 10, characterized in that said first liquid pervious layer and said second liquid pervious layer comprise substantially from 60% to 100% cellular fibres.

12. A contact-dry article according to any one of claim 4, or claims 6 to 11 when appended directly or indirectly to claim 4, characterized in that said bulk drainer layer comprises substantially from 50% to 100% cellular fibres.

13. A contactwry article according to any one of claims 6 or 6, or claims 7 to 12 when appended directly or indirectly to claim 5 or 6, characterized in that said third liquid pervious layer comprises substantially from 50% to 100% cellular fibres.

14. A contact-dry article according to any one of claim 2, or claims 3 to 13 when appended directly or indirectly to claim 2, characterized in that said first absorbent/drying layer comprises cellular fibres and/or hydrophobic fibres.

15. A contact-dry article according to any one of claims 3, or 4 to 14 when appended directly or indirectly to claim 3, characterized in that said second absorbent/drying layer comprises cellular fibres and/or hydrophobic fibres.

16. A contact-dry article according to any one of claims 8 to 15, characterized in that said cellular fibres comprise substantially from 35% to 85%.

17. A contact-dry article according to any one of claims 8 to 16, characterized in that said cellular fibres comprise cotton and/or hemp.

18. A contact-dry article according to any one of claims 1 to 17, characterized in that said first liquid pervious layer and said second liquid pervious layer each comprise substantially from 10% to 100% hydrophobic fibres.

19. A contact-dry article according to any one of claim 4, or claims 5 to 18 when appended directly or indirectly to claim 4, characterized in that said bulk drainer layer comprises substantially from 10% to 100% hydrophobic fibres.

20. A contact-dry article according to any one of claims 5 or 6, or claims 7 to 19 when appended directly or indirectly to claims 5 or 6, characterized in that said third liquid pervious layer comprises substantially from 10% to 100% hydrophobic fibre.

21. A contact-dry article according to any one of claims 8 to 20, characterized in that said hydrophobic fibres comprise substantially from 65% to 100%.

22. A contact-dry article according to any one of claims 8 to 21, characterized in that said hydrophobic fibres comprise polyester and/or acrylic.

23. A contact-dry article according to any one of claims 1 to 22, characterized in that said first liquid pervious layer is provided as a knitted fabric.

24. A contact-dry article according to any one of claims 5 or 6, or claims 7 to 22 when appended directly or indirectly to claims 5 or 6, characterized in that said third liquid pervious layer is provided as a knitted fabric.

25. A contact-dry article according to any one of claims 1 to 24, characterized in that said second liquid pervious layer is provided as a perforated fabric.

26. A contact-dry article according to any one of claims 2 to 25, characterized in that said first absorbent/drying layer comprises cotton.

27. A contact-dry article according to any one of claims 3, or 4 to 28 when appended directly or indirectly to claim 3, characterized in that said second absorbent/drying layer comprises cotton.

28. A contact-dry article according to any one of claim 4, or claims 5 to 27 when appended directly or indirectly to claim 4, characterized in that said bulk absorbent layer comprises compressible web material.

29. A contact-dry article according to any one of claim 4, or claims 5 to 28 when appended directly or indirectly to claim 4, characterized in that said bulk absorbent layer comprises terry towelling.

30. A contact-dry article according to any one of claims 1 to 29, characterized in that said liquid impervious layer comprises water-proof coated fabric.
